(19) United States
(12) Patent Application Publication
(10) Pub. No.: US 2018/0250225 A1
(43) Pub. Date: Sep. 6, 2018

(54) GUMMY COMPOSITION WITH IMPROVED STABILITY

(71) Applicant: Church & Dwight Co., Inc., Princeton, NJ (US)

(72) Inventors: Paige M. Appleton, Denver, CO (US); Jacob Adams, Somerville, NJ (US); Thomas G. Abene, Princeton, NJ (US)

(73) Assignee: Church & Dwight Co., Inc., Princeton, NJ (US)

(21) Appl. No.: 15/909,466

(22) Filed: Mar. 1, 2018

Related U.S. Application Data
(60) Provisional application No. 62/465,494, filed on Mar. 1, 2017.

(51) Int. Cl. A61K 9/00; A61K 35/644; A61K 47/02; A61K 47/26; A61K 47/36; A61K 47/42; A61K 47/46

(52) U.S. Cl. CPC A61K 9/0056; A61K 35/644; A61K 47/02; A61K 47/46; A61K 47/36; A61K 47/42; A61K 47/26

(57) ABSTRACT

The present disclosure describes a gummy dosage form including honey in an amount of 20% by dry weight or greater; fruit juice concentrate in an amount of 2% by dry weight or greater; a calcium source; one or more hydrating materials; and a hydrophilic long-chain polymer. The gummy dosage forms can further include additives such as flavorants, fiber, and pH-adjusters. The disclosure further comprises methods for preparing such gummy dosage forms.

### CROSS-REFERENCE TO RELATED APPLICATIONS

The present application claims priority to U.S. Provisional Patent Application No. 62/465,494, filed Mar. 1, 2017, which is incorporated herein by reference in its entirety.

### FIELD OF THE DISCLOSURE

The present disclosure relates to orally ingestible dosage forms and methods for making such dosage forms. The dosage forms can contain significant amounts of honey and fruit juice.

### BACKGROUND OF THE INVENTION

Oral dosing of many materials with desirable properties and functions can be problematic when provided in a chewable form because the intrinsic taste of such materials can be unpleasant, particularly in children and geriatric adults. The intrinsic bitterness of certain active pharmaceutical ingredients (APIs) in particular can present a major obstacle to the acceptance, compliance, and effectiveness of treatments including oral, chewable dosing.

Previous approaches to addressing the problem of poor palatability of certain materials have been based mainly on nullifying undesirable tastes using flavor additives, chemical chelation (e.g., using ion exchange resins and β-cyclodextrins) and physical encapsulation. These systems can be adapted into solid dosage forms or liquid-based formulations as solutions, suspensions, or multi-phase emulsions.

Gummy dosage forms are particularly effective for enabling compliant dosing in children, as well as geriatric adults, as these forms provide a palatable, chewable base, can incorporate APIs, and have low intrinsic taste response. However, while gummy dosage forms provide the basis for effective dosing of active ingredients to children (and geriatric patients), the preparation of stable gummy dosage forms including certain base ingredients (in particular, certain natural base ingredients) has been challenging. For example, the inclusion of honey in significant amounts commonly leads to stickiness, syneresis and/or other negative textural properties.

It would be beneficial to provide stable dosage forms with sufficient gummy properties incorporating natural ingredients, e.g., honey in significant amounts and to methods for providing such dosage forms.

### SUMMARY OF THE DISCLOSURE

The present disclosure provides chewable, gummy dosage forms that are adapted for the delivery of active ingredients to individuals, including those who may have difficulty swallowing conventional oral dosage forms (e.g., children and geriatric adults) and those who have an aversion to the taste of the active ingredients or have dosing fatigue to swallowing pills. The present disclosure provides formulations that comprise significant amounts of honey and fruit juice.

One aspect of the invention provides a gummy dosage form for oral administration, comprising: honey in an amount of 20% by dry weight or greater; fruit juice concentrate in an amount of 2% by dry weight or greater; a calcium source; one or more hydrating materials; and a hydrophilic long-chain polymer. The calcium salt can, in some embodiments, be a calcium salt, e.g., tricalcium phosphate. Tricalcium phosphate, where present, can be in an amount of about 2% by dry weight to about 15% by dry weight, about 2% by dry weight to about 8% by dry weight, about 4% by dry weight to about 8% by dry weight, or about 10% by dry weight to about 15% by dry weight.

In certain embodiments, the honey within the disclosed gummy dosage forms is present in an amount of at least 25% or at least 30% by dry weight, e.g., an amount of 30% to about 40% by dry weight. In certain embodiments, the fruit juice concentrate within the disclosed gummy dosage forms is present in an amount of about 2% to about 20% by dry weight, e.g., an amount of about 5% to about 15% by dry weight or about 8% to about 12% by dry weight. The composition of the hydrophilic long-chain polymer component can vary and, in some embodiments, the hydrophilic long-chain polymer comprises pectin and gelatin.

In addition to the components referenced above, the disclosed gummy dosage forms can comprise various additional components. For example, in some embodiments, the gummy dosage form further comprises sucrose. Advantageously, in certain embodiments, honey is present in a greater amount than sucrose, such that, e.g., the dry weight ratio of honey to sucrose is greater than 1:1. For example, the dry weight ratio of honey to sucrose can be about 1:1 to about 2:1 or about 1.2:1 to about 1.7:1. In some embodiments, the gummy dosage form further comprises one or more food-grade additives selected from the group consisting of flavorants, colorants, fiber, and pH-adjusters. The gummy dosage forms can contain active ingredients, e.g., one or more vitamins. The disclosed gummy dosage form, in some embodiments, comprises no corn syrup. In certain embodiment, the gummy dosage form is a sanded gummy dosage form, which further comprises a sanded coating comprising sucrose on the exterior thereof.

### DETAILED DESCRIPTION OF THE INVENTION

The invention now will be described more fully herein after through reference to various embodiments. These embodiments are provided so that this disclosure will be thorough and complete, and will fully convey the scope of the invention to those skilled in the art. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a,” “an,” and “the” include plural referents unless the context clear dictates otherwise.

The disclosure relates to gummy dosage forms for oral use that are suitable particularly for the delivery of active ingredients in a manner that is highly palatable and that provides compliance with dosing requirements for the active ingredients. The disclosure, in particular, relates to gummy dosage forms that comprise a significant amount of natural ingredients, i.e., ingredients that are obtained from, e.g., plant matter, and are not derived from synthetic processes. In particular, the gummy dosage forms disclosed herein contain a significant amount of honey and fruit juice and exhibit good physical stability, as will be described in further detail herein below. The disclosure also relates to methods of preparing such gummy dosage forms.

A “gummy” or “gummy dosage form” as used herein is understood to refer to a confectionary that can be defined by its compositional nature, as otherwise described herein, and also by its chewy texture and mouthfeel. Gummy bears, gummy worms, and other gummy candies are known in the art, and a person of ordinary skill in the art would understand the term “gummy” to refer to a composition having such texture and mouthfeel. It is noted that the gummy dosage forms disclosed herein may vary somewhat in texture and mouthfeel. All such textures and mouthfeels are intended to be included within the general definition of “gummy.” Further information on gummy product components and properties is provided, for example, in U.S. Patent Application Publication Nos. 2016/0296470 and 2016/0296474 to Romanoschi et al., which are incorporated herein by reference in their entireties.

The “active ingredient” included within the gummy dosage forms disclosed herein can be any compound, composition, or like material that may be included in a dosage form for delivery to an individual to achieve any one or more of a desired nutritional purpose, medicinal purpose, and therapeutic purpose. The types of active ingredients incorporated within the disclosed gummy dosage forms include, but are not limited to, vitamins, minerals, phytonutrients (e.g., carotenoids, flavonoids, resveratrol, and glucosinolates), fiber, fatty acids, amino acids, polypeptides, and botanicals. Further, non-limiting examples of materials that may be included as an active ingredient include APIs, and non-limiting examples of APIs include non-steroidal anti-inflammatory drugs (NSAIDs—e.g., ibuprofen, diclofenac, and naproxen), analgesics (e.g., acetaminophen, aspirin), antihistamines, decongestants, antitussives, expectorants, sleep aids, antibiotics, laxatives, anti-diarrheals, anthelmintics, and antacids. An active ingredient can include any plant-derived material that is safe for human consumption, including herbal extracts, botanical extracts, and the like. Other materials, such as prebiotics, probiotics, can also be used as an active ingredient. In some embodiments, an active agent according to the present disclosure may be classified as dietary supplement according to the Dietary Supplement Health and Education Act of 1994, whereby a dietary supplement is defined to mean a product (other than tobacco) intended to supplement the diet that bears or contains one or more of the following dietary ingredients: a vitamin, a mineral, an herb or other botanical, an amino acid, a dietary substance for use by man to supplement the diet by increasing the total dietary intake; or a concentrate, metabolite, constituent, extract, or combination of any of the aforementioned ingredients.

In certain embodiments, the active ingredient comprises a combination of such components, e.g., in the form of a multivitamin, comprising more than one vitamin. The relative amounts of the vitamins can vary and may be such that the resulting gummy dosage form is suited generally for adults (including specific formulations for men and women) or children. The specific active ingredients and amounts of a given dosage form can be designed for specific benefits, e.g., to promote immune health or to provide prenatal benefits. Certain active ingredients for inclusion within a multivitamin gummy dosage form as disclosed herein include any combination of two or more of Vitamin A, Vitamin C, Vitamin D3, Vitamin E, thiamin, riboflavin, niacin, Vitamin B6, folic acid, Vitamin B12, biotin, pantothenic acid, calcium, iodine, zinc, choline, and inositol.

The gummy dosage forms disclosed herein further comprise, in addition to the one or more active ingredients, one or more components, such as those components typically employed in gummy products, including, but not limited to, gellants, sweeteners, water, colorants, and flavorants. Gummy dosage forms generally comprise hydrocolloid systems, which can comprise, in some embodiments, one or more hydrophilic long-chain polymers, one or more hydrophilic bulking agents, and a hydrating material (water source). Optionally, the hydrocolloid system can include one or more further ingredients, such as pH modifiers, coloring agents, and/or flavoring agents. The gummy dosage form can further comprise one or more ions or ion sources, which can, in some embodiments, function to “set” the gummy form.

Hydrophilic, long-chain polymers useful in such hydrocolloid systems include, but are not limited to, long chain carbohydrates (e.g., polysaccharides) as well as various proteins. The hydrophilic, long-chain polymer preferably is configured to thicken and form a gel upon hydration (with or without heating). Non-limiting examples of hydrophilic, long-chain polymers that may be included in a hydrocolloid system for use within the gummy dosage forms disclosed herein include gelatin, pectin (including modified and unmodified forms of pectin), carrageenan, gellan gum, locust bean gum, gum arabic, xanthan gum, starch, methylcellulose, agar, konjac, alginates, and combinations thereof (including single, binary, tertiary, or quaternary blends).

In certain disclosed gummy dosage forms, at least a portion of the hydrocolloid system of the dosage forms comprises pectin. Pectin is a heteropolysaccharide that is rich in galacturonic acid. Pectin is available in both high methoxyl and low methoxyl forms, wherein the reference to “methoxyl” refers to the number of carboxyl groups on the pectin backbone that are methyl esters. Typically, “low-methoxyl” pectin is understood to comprise less than about 50% esterified carboxyl groups, and high-methoxyl, by contrast, is understood to comprise greater than about 50% esterified carboxyl groups. In certain embodiments herein, pectin is incorporated in high-methoxyl pectin form. Typically, the amount of pectin within the disclosed gummy dosage forms is less than about 15% by dry weight, e.g., in certain embodiments, less than about 10% by dry weight, less than about 5% by dry weight, less than about 2% by dry weight, or less than about 1% by dry weight, less than about 0.5% by dry weight, or less than about 0.25% by dry weight. An exemplary range of pectin content with respect to certain embodiments is about 0.1% to about 1.0% pectin by weight or about 0.1% to about 0.5% pectin by dry weight.

In certain disclosed gummy dosage forms, at least a portion of the hydrocolloid system of the dosage forms comprises gelatin. Typically, gelatin is included in an amount of at least about 2% by dry weight or at least about 5% by dry weight, e.g., about 2% to about 15% by dry weight or about 3% to about 10% by dry weight.

In certain embodiments, the hydrophilic long-chain polymer component in the disclosed gummy dosage forms comprises a combination of gelatin and pectin. The ratio of gelatin to pectin can vary, but in certain preferred embodiments, the overall amount of gelatin by dry weight in the disclosed gummy dosage forms is greater than the overall amount of pectin by dry weight. Certain exemplary weight ratios of these two components include about 1:1 to about 20:1, e.g., about 5:1 to about 20:1 or about 10:1 to about 20:1. Advantageous relative amounts of these components may be affected, in some embodiments, by the active ingredient(s) contained within the gummy dosage form.

The hydrophilic bulking agent component in the hydrocolloid systems typically includes one or more saccharides or saccharide derivatives. In exemplary embodiments, hydrophilic bulking agents can include oligofructose, dextrins, monosaccharides (e.g., fructose or glucose), disaccharides (e.g., platinose or sucrose), hydrogenated carbohydrates, also known as sugar alcohols (e.g., polyols, monosaccharide alcohols, disaccharide alcohols, or oligosaccharide alcohols) and syrups (e.g., glucose syrup or fructose syrup). The hydrophilic bulking agent further may be a synthetic material, such as soluble fiber (e.g., polydextrose).

According to the present invention, at least a portion of the hydrophilic bulking agent in the disclosed compositions comprises honey. Honey, as used herein, is a natural substance produced, e.g., from the sugary secretions of plants or insects. Honey generally contains glucose and fructose, as well as trace enzymes, vitamins, minerals, and amino acids. Honey produced by honey bees is the most common and commercially available honey and is advantageously incorporated within gummy dosage forms as disclosed herein. Honey is commonly classified by the floral source (or sources) of the nectar from which it was made. Honey comprising a mixture of two or more different honeys is referred to as “blended honey,” honey derived from the nectar of two or more types of flowers is referred to as “wildflower honey” or “polyfloral honey,” honey made primarily from the nectar of one type of flower is referred to as “monofloral honey.” Specific flowers from which honey can be derived include, but are not limited to, clover, orange blossom, blueberry, sage, tupelo, buckwheat, fireweed, mesquite, sourwood, thyme, thistle, heather, acacia, dandelion, sunflower, lavender, honeysuckle, lime, chestnut, clover, and cotton.

Honey is further classified by the United States Department of Agriculture based on its color (ranging from “water white” to “dark amber”) and quality (Grade A, Grade B, Grade C, or “Substandard”) and any such honeys can be used within the disclosed gummy dosage forms. Another distinguishing characteristic of honeys that can be used to classify different honeys is its appearance/form, which can be correlated with processing treatments to which it has been subjected. For example, raw honey is unprocessed or minimally processed and is not subjected to heat; crystallized honey comprises at least some solid crystals of honey (but can be returned to the liquid form by heating); strained honey has been filtered so as to remove, e.g., wax and other solid materials (allowing pollen, minerals, and enzymes to remain in the honey); filtered honey has been filtered, optionally using heat, to remove more materials, including fine particles, pollen grains, air bubbles, etc.; ultrasonicated honey has been processed by ultrasonication (typically resulting in the destruction of yeast cells and the inhibition of crystallization), pasteurized honey has been heated in a pasteurization process (which can affect the color, taste, and fragrance); creamed honey (also referred to as whipped, spun, churned, or set honey) is processed to produce a smooth, spreadable honey with many small crystals; and dried honey is processed to remove a substantial amount of the liquid therefrom to create granules.

Although any of the honey classes described herein above can be included within the disclosed gummy dosage form, in certain embodiments, the gummy dosage forms disclosed herein and, in particular, at least a portion of the hydrophilic bulking agent beneficially comprises pasteurized honey. Advantageously, the hydrophilic bulking agent in the disclosed gummy compositions comprises at least about 30% honey, at least about 40% honey, at least about 50% honey, at least about 55% honey, or at least about 60% honey, based on the dry weight of hydrophilic bulking agents present in the gummy dosage form.

In some embodiments, sugar (i.e., sucrose) is incorporated into the disclosed gummy dosage forms as a further hydrophilic bulking agent. For example, in some embodiments, the hydrophilic bulking agent in the disclosed gummy compositions can comprise less than about 70%, less than about 60%, less than about 50% sucrose, with representative amounts being about 5% to about 70% by dry weight of sucrose, e.g., about 10% to about 60% by dry weight of sucrose, about 20% to about 50% by dry weight of sucrose, or about 30% to about 50% by dry weight of sucrose, or about 35% to about 45% by dry weight of sucrose. Although other hydrophilic bulking agents can be incorporated within the gummy dosage forms, in some embodiments, the hydrophilic bulking agent comprises less than about 50%, less than about 40%, less than about 30%, less than about 20%, less than about 10%, less than about 5%, less than about 1%, or 0% by weight of monosaccharides (e.g., fructose or glucose) from a source other than the honey. In some embodiments, the hydrophilic bulking agent comprises no high fructose corn syrup.

As such, certain embodiments provide gummy dosage forms wherein the hydrophilic bulking agent comprises a combination of honey and sucrose. The honey and sucrose are present in varying dry weight ratios with respect to one another, wherein the honey is typically present in a higher dry weight percentage than the sugar. Thus, in some embodiments, they may be present in a dry weight ratio of greater than 1:1 honey: sucrose, such as a dry weight ratio of about 1:1 to 2:1, e.g., about 1.2:1 to about 1.7:1. In some embodiments, the total sucrose incorporated within the disclosed gummy dosage forms is less than about 40% by dry weight, less than about 30% by dry weight, or less than about 25% by weight (e.g., about 20% to about 30% or about 20% to about 25% by dry weight).

The relative amounts of the components used in the hydrocolloid system in the oral gummy dosage forms provided herein can vary. Overall, the hydrophilic bulking agent is, in preferred embodiments, included within the disclosed gummy dosage forms in an amount of about 20 to about 80% by dry weight. In some embodiments, the disclosed gummy dosage form can comprise about 40% to about 70%, or about 50% to about 60% by dry weight of the hydrophilic bulking agents (particularly honey and sucrose). As such, honey is typically included within the disclosed gummy dosage forms in a significant amount overall, e.g., in an amount of about 10% by dry weight or more, about 15% by dry weight or more, about 20% by dry weight or more, or about 30% by dry weight or more. Exemplary weight ranges of honey include about 5% to about 50%, about 10% to about 50%, about 20% to about 40%, or about 30% to about 35% by dry weight, based on the gummy dosage form in its entirety. Although other hydrophilic bulking agents can be incorporated within the gummy dosage forms, in some embodiments, the hydrophilic bulking agent comprises less than about 50%, less than about 40%, less than about 30%, less than about 20%, less than about 10%, less than about 5%, less than about 1%, or 0% by weight of monosaccharides (e.g., fructose or glucose) from a source other than the honey. In some embodiments, the hydrophilic bulking agent comprises no glucose or fructose and, in preferred embodiments, the hydrophilic bulking agent comprises no high fructose corn syrup.

Further, the disclosed gummy dosage forms provided herein comprise one or more fruit or vegetable juice concentrates. Fruit and vegetable juice concentrates include juice from any one or more fruits and/or vegetables, which has been processed so as to remove at least a portion of the liquid therefrom. Such fruit juice concentrates are typically concentrated to a Degrees Brix of about 60 or greater or about 65 or greater. Brix is a unit of measurement of sugar content in an aqueous solution and 1 percent or 1 degree brix (Bx) is defined as 1 gram of sucrose in 100 grams of solution. Brix measurements are generally made by measuring the specific gravity of the solution/slurry using various instruments including, but not limited to, hydrometer, refractometer, pycnometer, or U-tube meter. The specific gravity can be converted to Bx, for example, using the Brix Table maintained by the National Institute of Standards and Technology.

Fruit juice concentrates include, but are not limited to, juices from apple, apricot, banana, blackberry, black currant, black raspberry, blueberry, boysenberry, grape, grapefruit, cranberry, cherry, elderberry, kiwi, guava, mango, passion fruit, peach, nectarine, pear, plum, pomegranate, red currant, red raspberry, blue raspberry, strawberry, watermelon, lime, lemon, orange, and pineapple. Vegetable juice concentrates include, but are not limited to, juices from butternut squash, tomato, celery, cucumber, kale, carrot, pumpkin, beet, rhubarb, and combinations thereof. The amount of juice concentrate incorporated within the disclosed gummy dosage forms can vary, but is typically at least about 2% by dry weight, such as about 2% by dry weight to about 20% by dry weight, e.g., about 5% to about 15% or about 8% to about 12% by dry weight.

As such, gummy dosage forms disclosed herein advantageously comprise a combination of honey and one or more juice concentrates, and in particular embodiments, honey, sucrose, and fruit juice concentrate. In some embodiments, the gummy dosage form comprises little to no “added sugars” other than sucrose. “Added sugars” as used herein refers to sugars other than sugars from natural sources (such as the sugars in the honey and the sugars in the juice concentrate), and includes such sugars added in solid or liquid, e.g., syrup form). As such, in certain embodiments, sucrose is the only added sugar in the gummy dosage forms disclosed herein. Various embodiments include gummy dosage forms comprising no more than about 10%, no more than about 5%, no more than about 2%, no more than about 1%, or no added sugar other than sucrose. In particular embodiments, the gummy dosage forms provided herein comprise no corn syrup.

The hydrating material (water source) of the hydrocolloid systems can include any variety of materials configured to donate water to the hydrophilic, long-chain polymer. The hydrating material particularly can be substantially pure water; however, the hydrating material may be an aqueous composition including one or more additives, such as a syrup, a fruit juice, or a flavoring liquid.

The disclosed gummy dosage forms also typically comprise a calcium source, e.g., tricalcium phosphate (TCP) or calcium carbonate. Other components that serve a similar function within the gummy dosage forms can, in some embodiments, be used in place of or in combination with the TCP or calcium carbonate. In some embodiments, tricalcium phosphate is employed, and is present within the disclosed gummy dosage forms in an amount of at least about 2% by dry weight, e.g., about 2% to about 15%, e.g., about 2% to about 8%, about 4% to about 8% by dry weight or about 10% to about 15%. In some embodiments, this component helps in decreasing the “stickiness” associated with certain gummy dosage form compositions.

In some embodiments, a pH modifier included in the hydrocolloid system particularly can be a buffer or acidifier. Non-limiting examples of buffer and/or acidic materials that can be used include citric acid, sodium citrate, malic acid, lactic acid, tartaric acid, fumaric acid, phosphoric acid, ascorbic acid, sodium bisulfate, and combinations thereof. Flavoring agents can by natural or artificial and include, but are not limited to, citric acid, tartaric acid, artificial sweeteners (e.g., acesulfame potassium, aspartame, neotame, saccharine, and sucralose); salts (e.g., sodium chloride); plant extracts (e.g., vanilla, luo han guo); vegetable juice (e.g., carrot concentrate), pulp, and/or extracts; fruit juice, pulp, zest, and/or extracts (e.g., strawberry, raspberry, blackberry, blueberry); nuts; seeds; warm sensation materials; cool sensation materials; tingling sensation materials; and essential oils.

In a non-limiting example, a gummy dosage form is provided which comprises about 25% to about 35% honey, about 20% to about 25% sucrose, about 8% to about 14% fruit juice concentrate, about 2% to about 20% tricalcium phosphate, about 2% to about 10% gelatin, and about 0.1% to about 5% pectin, based on the total dry weight of the gummy dosage form. The gummy dosage forms provided according to the present disclosure thus generally comprise a significant amount of honey in addition to one or more active ingredients, as described herein above.

In certain embodiments of the present disclosure, a gummy dosage form is provided wherein the active ingredients are substantially homogenously distributed throughout the dosage form. In particular, the active ingredient is, in some embodiments, substantially homogeneously distributed throughout the hydrocolloid system. The gummy dosage forms may comprise an outer coating or may be uncoated. The gummy dosage forms can be provided in various sizes, shapes, and total weight. Exemplary gummy dosage forms as disclosed herein can be provided with masses including, but not limited to, masses in the range of about 1 g to about 6 g, or about 2 g to about 5 g. The gummy dosage forms can be generally homogeneous with respect to all components thereof or can include one or more components in a non-homogeneous association with remaining components. For example, a gummy dosage form may be provided wherein one or more components is only partially blended into the composition, e.g., so as to produce the effect of a visual “swirl” of colorant and/or flavorant on and/or within the composition. Such “swirls” and other patterned non-homogeneities of colorants, flavorants, and/or other components are intended to be encompassed by the present disclosure.

The gummy dosage forms provided herein generally can be characterized as being elastic or viscoelastic materials, and can be described as substantially chewable. A “chewable” dosage form, while capable of being swallowed whole, is configured specifically for chewing prior to swallowing. As such, a chewable dosage form is specifically distinguishable from a non-chewable dosage form, such as a vitamin tablet or capsule that is intended to be swallowed whole. In some embodiments, the term chewable can thus mean that the dosage form is intended to be retained in the mouth of the consumer for a period of time prior to swallowing, during which time the dosage form may undergo a change in structure that facilitates ease of swallowing. The chewable dosage form may thus be reduced to smaller pieces through mastication. In some embodiments, the chewable dosage form may be configured to at least partially dissolve within the mouth of the consumer. As such, the chewable dosage form may also be dissolvable and may thus be referred to as a “melt-away” form.

It is understood that the oral dosage forms of the present disclosure may be configured for undergoing changes under various mouth conditions. Discussion herein of “mouth conditions” can relate to one or more characteristics (in any combination) associated with the presence of an item in the mouth of an individual. For example, mouth conditions can include any combination of temperature, moisture, and pH typically found in the mouth of a human as well as the shear, compression, and other mechanical forces that may be applied by the teeth during chewing. Mouth conditions can particularly relate to being in contact with saliva. In some embodiments, mouth conditions can particularly mean contact with saliva at the temperature and pH typically present in the human mouth.

Advantageously, the disclosed dosage forms comprise a sanded coating on at least a portion, and preferably on all exposed surfaces of the gummy dosage form. Sanded coatings and methods for applying such coatings are generally known in the art. Sanded coatings are typically particulate materials (e.g., comprising particulate/granulated sugar, particulate sugar polyols, or combinations thereof) that, in the context of the disclosed gummy dosage form, are coated on and adhere to the surface thereof by electrostatic forces and/or mechanical adherent properties.

The composition of the sanded coating can vary and, in certain embodiments, is primarily sucrose, e.g., about 50% by weight or more sucrose, about 60% by weight or more sucrose, about 70% by weight or more sucrose, about 80% by weight or more sucrose, or about 90% by weight or more sucrose, e.g., about 80-95% by weight sucrose. The remainder of the sanded coating can include, e.g., flavorants or pH adjusters as disclosed herein above, which may, in some embodiments, modify the specific taste characteristics (e.g., the inclusion of certain acids can provide a sour flavor on the exterior of the gummy dosage form). Overall, the sanded coating, where present, comprises a relatively small percentage of the sanded gummy dosage form, e.g., about 20% or less by weight of the sanded gummy dosage form or about 10% or less by weight of the sanded gummy dosage form.

The disclosed gummy dosage forms advantageously exhibit high physical stability. Stability in this sense is understood to refer to, e.g., minimal stickiness and minimal syneresis. Such physical characteristics can be evaluated in various ways, e.g., by physical evaluation, which preferably indicates little to no change in the shape of the gummy dosage form over a period of days, e.g., for about 10 days or more, about 30 days or more, about 60 days or more, about 6 months or more, after a year or more, or after 2 years or more. The combination of components and amounts identified herein (e.g., honey, sucrose, fruit juice concentrate, and calcium source) uniquely provide a highly stable dosage form wherein honey is a principal component. Advantageously, the disclosed dosage forms exhibit extended flavor release and, texturally, minimal toothstick.

In preferred embodiments, a plurality of the disclosed gummy dosage forms “flow freely,” i.e., do not stick to one another to any significant extent. As such, in preferred embodiments, the disclosed gummy dosage forms are not difficult to extract from a container due to interaction/sticking between multiple such dosage forms.

Certain exemplary gummy dosage forms as disclosed herein comprise the components in amounts as follows in Table 1:

TABLE 1
Exemplary Gummy Dosage Form Formulation

| | | | | | Fruit Juice | Calcium |
|---|---|---|---|---|---|---|
| Gelatin | Pectin | Honey | Sucrose | Water | Concentrate | (TCP) |
| 0-15% | 0-5% | 29-36% | 15-78% | 3-19% | 3-30% | ~5-15% |

*At least some gelatin and/or pectin must be included to provide a gummy dosage form.

The term “dry weight” as used herein is based on and/or means the total weight of the finished product (i.e., the gummy dosage form). As such, all reported amounts and percentages are to be construed accordingly.

The disclosure also provides methods for preparing gummy dosage forms. Specifically, the disclosed methods involve steps of preparing a hydrocolloid system slurry, optionally including one or more active ingredients, heating the hydrocolloid system slurry to thicken the slurry (and specifically to achieve a particular brix level) and subsequently adding the one or more additional ingredients thereto to provide a gummy dosage form.

Generally, a slurry comprising the hydrocolloid system (including the hydrophilic long-chain polymer, hydrophilic bulking agent, water, and other optional components as disclosed herein above) is prepared. The components can be combined in various orders. For example, in one embodiment, at least a portion of the hydrophilic long-chain polymer is added to water comprising sodium citrate (e.g., at elevated temperature) to give a first mixture. In one particular embodiment, sodium citrate, a foam suppressor, and hot water are combined and heated to 180° F., and pectin, water, and gelatin are added thereto (giving a first mixture). This first mixture can be stirred and, optionally heated to allow for dissolution of the hydrophilic long-chain polymer. To this first mixture, certain remaining components, including honey, fruit juice concentrate, and tricalcium phosphate (or maltodextrin), are then added. Advantageously, such components can be pre-combined and heated at elevated temperature (e.g., 180° F.) and this second mixture is added to the first mixture. Subsequently, the sugar and certain active ingredients are then added.

The resulting slurry comprising the hydrocolloid system is heated/cooked to the desired concentration. The temperature can vary, and in one particular embodiment is about 230-250° F., e.g., about 240° F.). Generally, the desired concentration can be defined by the brix level of the slurry, which can be monitored and measured as described herein below. In some embodiments, the hydrocolloid slurry, just prior to addition of the active ingredient(s), has a Brix of at least about 50%, at least about 60%, at least about 70%, e.g., about 60-80% Brix or about 70-80% Brix (e.g., 79% Brix).

The active ingredient(s) to be incorporated within a gummy dosage form can be added to the slurry prior to cooking (i.e., when the slurry is in less concentrated form) to ensure homogeneity of the resulting product or can be added after cooking the slurry to form a thickened slurry (i.e., when the slurry has achieved the brix levels referenced above). In some embodiments, solid active ingredients are added prior to cooking, and liquid active ingredients are added after cooking. Typically, colors, flavors, and acids are also added as a post-dose after cooking.

The active ingredient-containing, thickened slurry is deposited into molds and cooled therein to set the final, desired shape when released from the molds to provide gummy dosage forms. The time required to achieve the gummy dosage forms after depositing into the molds can vary. Typically, sufficient setting of the slurry is achieved in the molds within an hour at ambient conditions (room temperature and ambient pressure) and sufficient drying of the slurry is achieved in the molds within about 72 hours, after which time the gummy dosage forms can be removed from the molds. It is noted that certain forms, e.g., those deposited on a silicone mold, require little to no drying time (e.g., less than about 1 hour). Advantageously, the gummy dosage forms exhibit sufficient integrity to remain in the desired form (without flowing) after removal from the molds. In some embodiments, the dosage forms removed from the molds can be processed, e.g., by applying oil or anti-sticking agents thereto, or by applying a coating as known in the art thereto. In particular, as referenced herein above, a sanded coating is advantageously applied to the disclosed gummy dosage forms following removal from the molds.

Embodiments of the present disclosure are further illustrated by the following examples, which are set forth to illustrate the presently disclosed subject matter and are not to be construed as limiting.

### EXPERIMENTAL

### Example 1: Particular Formulations

Certain specific formulations are presented herein below, which are understood to be exemplary, rather than limiting of the present invention. The formulations do not include active ingredient/vitamin components or certain non-active components (e.g., colors, acidulants, etc).

TABLE 2
Gummy Dosage Form A (active ingredient/vitamin
components not included)

| Ingredient | Amount (% by dry weight) |
|---|---|
| Honey | 33.85 |
| Sugar (sucrose) | 22.98 |
| Fruit juice concentrate | 10.87 |
| Tricalcium phosphate | 5.00 |
| Gelatin | 7.00 |
| Pectin/sugar^{A} | 0.25 |
| Sodium citrate | 0.01 |
| Flavorant | 1.07 |
| Sanded coating^{B} | 9.50 |

^{A} 1:1 pectin: sugar blend (by weight)

^{B} Includes sucrose and encapsulated citric and fumaric acids

The finished product moisture of the gummy dosage form of Table 2 was 3.72%.

TABLE 3
Gummy Dosage Form B (active ingredient/vitamin
components not included)

| Ingredient | Amount (% by dry weight) |
|---|---|
| Honey | 31.34 |
| Sugar (sucrose) | 21.28 |
| Fruit juice concentrate | 10.06 |
| Tricalcium phosphate | 5.00 |
| Gelatin | 8.00 |
| Pectin/sugar^{A} | 0.25 |
| Sodium citrate | 0.01 |
| Flavorant | 0.96 |
| Sanded coating^{B} | 9.50 |

The finished product moisture of the gummy dosage form of Table 3 was 4.23%.

TABLE 4
Gummy Dosage Form C (active ingredient/vitamin
components not included)

| Ingredient | Amount (% by dry weight) |
|---|---|
| Honey | 29.62 |
| Sugar (sucrose) | 24.19 |
| Fruit juice concentrate | 9.04 |
| Tricalcium phosphate^{C} | 14.89 |
| Gelatin | 4.00 |
| Pectin/sugar^{A} | 1.60 |
| Flavorant | 0.65 |
| Sanded coating^{B} | 5.50 |

^{C} The tricalcium phosphate in this dosage form served also as an active ingredient, to provide calcium

The finished product moisture of the gummy dosage form of Table 4 was 7.96%.

TABLE 5
Gummy Dosage Form D (active ingredient/vitamin
components not included)

| Ingredient | Amount (% by dry weight) |
|---|---|
| Honey | 35.90 |
| Sugar (sucrose) | 24.38 |
| Fruit juice concentrate | 11.53 |
| Tricalcium phosphate | 5.00 |
| Gelatin | 7.00 |
| Pectin/sugar^{A} | 0.25 |
| Sodium citrate | 0.01 |
| Flavorant | 0.96 |
| Sanded coating^{B} | 9.50 |

The finished product moisture of the gummy dosage form of Table 5 was 4.05%.

TABLE 6
Gummy Dosage Form E (active ingredient/vitamin
components not included)

| Ingredient | Amount (% by dry weight) |
|---|---|
| Honey | 35.77 |
| Sugar (sucrose) | 24.54 |
| Fruit juice concentrate | 11.49 |
| Tricalcium phosphate | 5.00 |
| Gelatin | 7.00 |
| Pectin/sugar^{A} | 0.25 |
| Sodium citrate | 0.01 |
| Flavorant | 1.08 |
| Sanded coating^{B} | 9.50 |

The finished product moisture of the gummy dosage form of Table 6 was 3.16%.

TABLE 7
Gummy Dosage Form F (active ingredient/vitamin
components not included)

| Ingredient | Amount (% by dry weight) |
|---|---|
| Honey | 33.51 |
| Sugar (sucrose) | 22.75 |
| Fruit juice concentrate | 10.76 |
| Tricalcium phosphate | 5.00 |
| Gelatin | 7.00 |
| Pectin/sugar^{A} | 0.25 |
| Sodium citrate | 0.01 |
| Flavorant | 1.11 |
| Sanded coating^{B} | 9.50 |

The finished product moisture of the gummy dosage form of Table 7 was 3.81%.

TABLE 8
Gummy Dosage Form G (active ingredient/vitamin
components not included)

| Ingredient | Amount (% by dry weight) |
|---|---|
| Honey | 33.31 |
| Sugar (sucrose) | 22.87 |
| Fruit juice concentrate | 10.70 |
| Tricalcium phosphate | 5.00 |
| Gelatin | 7.00 |
| Pectin/sugar^{A} | 0.25 |
| Sodium citrate | 0.01 |
| Flavorant | 0.90 |
| Sanded coating^{B} | 9.50 |

The finished product moisture of the gummy dosage form of Table 8 was 3.77%.

TABLE 9
Gummy Dosage Form H (active ingredient/vitamin
components not included)

| Ingredient | Amount (% by dry weight) |
|---|---|
| Honey | 32.46 |
| Sugar (sucrose) | 22.40 |
| Fruit juice concentrate | 10.71 |
| Tricalcium phosphate | 4.96 |
| Gelatin | 6.25 |
| Pectin/sugar^{A} | 0.50 |
| Sodium citrate | 0.01 |
| Flavorant | 1.06 |
| Sanded coating^{B} | 8.68 |

The finished product moisture of the gummy dosage form of Table 9 was 5.25%.

TABLE 10
Gummy Dosage Form I (active ingredient/vitamin
components not included)

| Ingredient | Amount (% by dry weight) |
|---|---|
| Honey | 32.11 |
| Sugar (sucrose) | 22.11 |
| Fruit juice concentrate | 10.58 |
| Tricalcium phosphate | 5.28 |
| Gelatin | 6.25 |
| Pectin/sugar^{A} | 0.50 |
| Sodium citrate | 0.01 |
| Flavorant | 1.10 |
| Sanded coating^{B} | 8.68 |

The finished product moisture of the gummy dosage form of Table 10 was 5.13%.

TABLE 11
Gummy Dosage Form I (active ingredient/vitamin
components not included)

| Ingredient | Amount (% by dry weight) |
|---|---|
| Honey | 32.19 |
| Sugar (sucrose) | 22.39 |
| Fruit juice concentrate | 10.71 |
| Tricalcium phosphate | 5.00 |
| Gelatin | 5.00 |
| Pectin/sugar^{A} | 0.49 |
| Sodium citrate | 0.01 |
| Flavorant | 1.46 |
| Sanded coating^{B} | 6.54 |

The finished product moisture of the gummy dosage form of Table 11 was 4.26%.

### Example 2: Evaluation of Particular Forms

Gummy dosage forms comprising a multi-vitamin mixture intended for children (a blue raspberry-flavored form and a strawberry form) were evaluated by children participants in comparison to a commercially available comparative gummy dosage form. The participants ranked the gummy dosage forms falling within the present disclosure as significantly higher in “overall liking,” and “would ask parent to buy” opinion polls, and greater than 80% of participants indicated that they liked these gummy dosage forms “the same,” or “better than,” their current multivitamin (whereas only 41% of participants ranked the commercially available comparative gummy dosage form as being the same or better than their current multivitamin). The overall flavor of the present gummy dosage forms was considered by most participants (72 or 73%) to be “just about right,” whereas the overall flavor of the commercially available comparative gummy dosage form was considered by only 31% to be “just about right,” with the majority (58%) finding the overall flavor to be too strong. The sweetness, sourness, and sugar coating properties of the disclosed gummy dosage form were also found to be superior to the commercially available comparative product (with less than 6% of participants finding the sweetness to be “too weak,” about 15% or less of participants finding the sourness to be “too strong,” and less than 9% of participants finding the sugar coating to be “too weak.” Participants also evaluated the texture of the products, with at least 70% considering the disclosed gummy dosage forms to be “just right,” and almost 40% considering the commercially available commercial product to be “too soft.” A high percentage (62-63% of participants) considered the disclosed gummy dosage form to be “not sticky,” whereas only 49% of participants considered the commercially available comparative gummy dosage form to exhibit this characteristic. Overall, the “mouthfeel” of the disclosed gummy product was considered to be “really good/good” to about 70-80% of participants, whereas the mouthfeel of the commercially available comparative product was considered to be “really good/good” to only 37% of participants. The “aftertaste” of the disclosed gummy product was considered to be “really good/good” to about 70-80% of participants, whereas the mouthfeel of the commercially available comparative product was considered to be “really good/good” to only 29% of participants.

1. A gummy dosage form for oral administration, comprising:
honey in an amount of 20% by dry weight or greater;
fruit juice concentrate in an amount of 2% by dry weight or greater;
a calcium source;
one or more hydrating materials; and
a hydrophilic long-chain polymer.

2. The gummy dosage form of claim 1, wherein the calcium source is a calcium salt.

3. The gummy dosage form of claim 1, wherein the calcium source is tricalcium phosphate.

4. The gummy dosage form of claim 3, wherein the tricalcium phosphate is present in an amount of about 2% by dry weight to about 8% by dry weight.

5. The gummy dosage form of claim 3, wherein the tricalcium phosphate is present in an amount of about 4% by dry weight to about 8% by dry weight.

6. The gummy dosage form of claim 3, wherein the tricalcium phosphate is present in an amount of about 10% by dry weight to about 15% by dry weight.

7. The gummy dosage form of claim 1, wherein the honey is present in an amount of at least 25% by dry weight.

8. The gummy dosage form of claim 1, wherein the honey is present in an amount of at least 30% by dry weight.

9. The gummy dosage form of claim 1, wherein the honey is present in an amount of 30% to about 40% by dry weight.

10. The gummy dosage form of claim 1, wherein the fruit juice concentrate is present in an amount of about 2% to about 20% by dry weight.

11. The gummy dosage form of claim 1, wherein the fruit juice concentrate is present in an amount of about 5% to about 15% by dry weight.

12. The gummy dosage form of claim 1, wherein the hydrophilic long-chain polymer comprises pectin and gelatin.

13. The gummy dosage form of claim 1, further comprising sucrose.

14. The gummy dosage form of claim 13, wherein the dry weight ratio of honey to sucrose is greater than 1:1.

15. The gummy dosage form of claim 13, wherein the dry weight ratio of honey to sucrose is about 1:1 to about 2:1.

16. The gummy dosage form of claim 13, wherein the dry weight ratio of honey to sucrose is about 1.2:1 to about 1.7:1

17. The gummy dosage form of claim 1, wherein the gummy dosage form comprises no corn syrup.

18. The gummy dosage form of claim 1, further comprising one or more food-grade additives selected from the group consisting of flavorants, colorants, fiber, and pH-adjusters.

19. The gummy dosage form of claim 1, further comprising one or more vitamins.

20. The gummy dosage form of claim 1, further comprising a sanded coating comprising sucrose.
